(19) United States
(12) Patent Application Publication
(10) Pub. No.: US 2014/0058306 A1
(43) Pub. Date: Feb. 27, 2014

(54) ADJUSTABLE LENGTH ORTHOTIC DEVICE AND METHOD FOR USING THE SAME

(71) Applicant: Bio Cybernetics International, Inc., La Verne, CA (US)

(72) Inventor: ED BANNISTER, Riverside, CA (US)

(73) Assignee: Bio Cybernetics International, Inc.

(21) Appl. No.: 13/653,808

(22) Filed: Oct. 17, 2012

Related U.S. Application Data
(60) Provisional application No. 61/692,650, filed on Aug. 23, 2012.

(51) Int. Cl. A61F 5/02

(52) U.S. Cl. CPC A61F 5/02
USPC 602/19

(57) ABSTRACT

Described herein is an adjustable length orthotic device and a method for adjusting the length of said orthotic device. The adjustable length orthotic device can comprise a center brace segment having first and second pockets, and first and second end pieces for inserting into the first and second pockets, respectively. The subject invention addresses the problem of a patient being unable to adjust the length of his or her orthotic device in response to changes in the patient's size. The subject invention also addresses the problem of prior art adjustable length orthotic devices that are either overly complicated and cumbersome or have securing means on the outside of the brace that may snag on foreign objects, causing the brace to become undone or causing further injury to the patient. In one embodiment, the length of the orthotic device is adjusted by securing the two end segments inside the two pockets.

### CROSS-REFERENCE TO RELATED APPLICATION

Priority is claimed to provisional patent application no. 61/692,650, filed on Aug. 23, 2012, and titled “Adjustable Length Orthotic Device and Method for Using the Same.”

### BACKGROUND OF THE INVENTION

The present invention relates generally to orthotic devices and methods for using an orthotic device, and more particularly to a body brace for medical or recuperative purposes in which the length of the body brace can be adjusted to fit a wide range of users.

An orthotic device or orthosis (commonly known as a brace or splint) is an orthopedic device that is typically applied to a limb or the body. Among other things, the purpose can be to provide support, protection, pain reduction, or replacement of lost function.

In this regard, a common method of alleviating pain in people suffering from back pain or injuries and promoting healing in post-operative back surgery patients is to stabilize the spine by means of an orthosis, such as a brace. There are a large variety of braces available depending on the diagnosis and physical needs of the individual. These devices include a multitude of construction materials and designs which can be snugly fitted around the patient's trunk and peripheral area such as the cervical and pelvic regions. Such braces are effective in achieving spinal stability if worn properly and consistently.

Patients of many different sizes may need to use back braces. As such, back braces of different sizes are required to fit this variety of patients. Prior art back braces have dealt with this issue by providing a single back brace that is generally longer than most patients would need, and then cutting off the ends of the brace so that the brace is of a suitable length for the patient. However, this approach restricts the patient to a single-sized brace that cannot be changed in response to changes in the patient's size, e.g., if the patient gains weight. If the brace becomes too small for the patient to use, then they must be issued a new brace that is again cut to fit their current size.

Prior art back braces have provided adjustable length braces. These adjustable length braces have incorporated several different methods of adjusting the length of the brace. In one method, the brace includes a middle brace piece, and two end brace pieces, and a plurality of intermediate pieces that can be inserted between the middle brace piece and the two end pieces to adjust the length of the brace. This approach generally does not allow for small adjustments in length to the brace, as it is impractical to have very small intermediate pieces. Thus, this method requires that any size adjustment be made according to fixed units of length. For example, if each intermediate piece is two inches long, then the patient is unable to adjust the back brace to be just one inch or half an inch longer. Perhaps even more importantly, with this method of length adjustment, a large number of pieces and connections are required to adjust the length of the brace, making the brace somewhat complicated to use, potentially discouraging the patient from properly using the brace.

Another approach to adjusting the length of a brace includes length adjustment means, such as a plurality of straps, connecting a middle brace piece to two end brace pieces. The straps can be pulled tighter to shorten the brace or loosened to lengthen the brace. The length of the brace is then set by securing the straps using some attachment means, such as hook-and-loop fastener. This method avoids the issue of requiring numerous pieces to adjust the brace's length. However, a common problem with this method of adjusting the brace is that the straps must be secured somewhere along the outside of the brace. Otherwise, the straps would have to be secured somewhere along the inside of the brace, which is supposed to be snug against the patient, thereby causing discomfort to the patient. Having the straps on the outside of the brace creates the potential for the straps to get caught on objects as the patient walks past, causing the straps to become undone and loosening the brace, or potentially even causing further injury to the patient due to the force resulting from the straps getting caught on a foreign object.

It can readily be appreciated that there is a need for a back brace that is able to be adjusted in length in response to the needs of the patient, e.g., if the patient's size changes. It can further be appreciated that there is also a need for an adjustable length brace that does not include a large number of pieces to adjust the brace's length and does not have loose parts on the outside of the brace that could get caught on objects as the patient passes by them. The present invention fulfills these needs and provides further related advantages.

### SUMMARY OF THE INVENTION

The present invention is embodied in an adjustable length orthotic device comprising a center brace segment having two pockets for receiving two end segments. In one embodiment, the subject invention addresses the problem of a patient having to purchase a new orthotic device when they outgrow their current device, by allowing a single orthotic device to be adjusted in length.

In one embodiment, the adjustable length orthotic device comprises a center brace segment having a first pocket and a second pocket, a first end segment, and a second end segment. The first end segment is configured to be inserted into the first pocket, and the second end segment is configured to be inserted into the second pocket. The length of the adjustable length orthotic device may be adjusted by adjusting the respective positions of the first and second end segments inside the first and second pockets, respectively.

In another embodiment, the adjustable length orthotic device further comprises a securing means for securing the first and second end segments within the first and second pockets, respectively. The securing means may be hook and loop fastener.

In one embodiment, the adjustable length orthotic device is a back brace.

The invention is also embodied in a method for adjusting the length of an adjustable length orthotic device. In one embodiment, the method comprises the steps of obtaining an adjustable length orthotic device comprising a center brace segment having a first pocket and a second pocket, a first end segment that is configured to be inserted into the first pocket, and a second end segment that is configured to be inserted into the second pocket; inserting the first end segment into the first pocket; and inserting the second end segment into the second pocket.

In another embodiment, the adjustable length orthotic device obtained in the method may also comprise a securing means for securing the first and second end segments within the first and second pockets, respectively, and the method further comprises the steps of releasably securing the first end segment within the first pocket at a first position, and releasably securing the second end segment within the second pocket at a second position. The securing means may be hook and loop fastener, and the orthotic device may be a back brace.

In a further embodiment, the method may further comprise the steps of unsecuring the first end segment from the first pocket, adjusting the first end segment's position within the first pocket, and re-securing the first end segment within the first pocket at a first adjusted position that is different from the first position (i.e., the first end segment's initial secured position within the first pocket), and then performing the same steps using the second end segment within the second pocket such that the second pocket is re-secured within the second pocket at a second adjusted position that is different from the second end segment's initial secured position within the second pocket.

These and other features and advantages of the invention should become more readily apparent from the detailed description of the preferred embodiments set forth below taken in conjunction with the accompanying drawings, which illustrate, by way of example, the principles of the invention.

### BRIEF DESCRIPTION OF THE DRAWINGS

Embodiments of the present invention will now be described, by way of example only, with reference to the following drawings.

FIG. 1A is a front elevation view of an adjustable length orthotic device having pockets for insertion of end segments with the end segments inserted into the pockets, in accordance with an embodiment of the present invention.

FIG. 1B is a front elevation view of the adjustable length orthotic device of FIG. 1 with the end segments pulled further out so as to increase the length of the orthotic device.

FIG. 2 is a front elevation view of the adjustable length orthotic device of FIG. 1 with the end segments pulled out of the pockets.

FIG. 3 is a side perspective view of the orthotic device of FIG. 1 with the end segments inserted into the pockets.

### DETAILED DESCRIPTION OF THE PREFERRED EMBODIMENTS

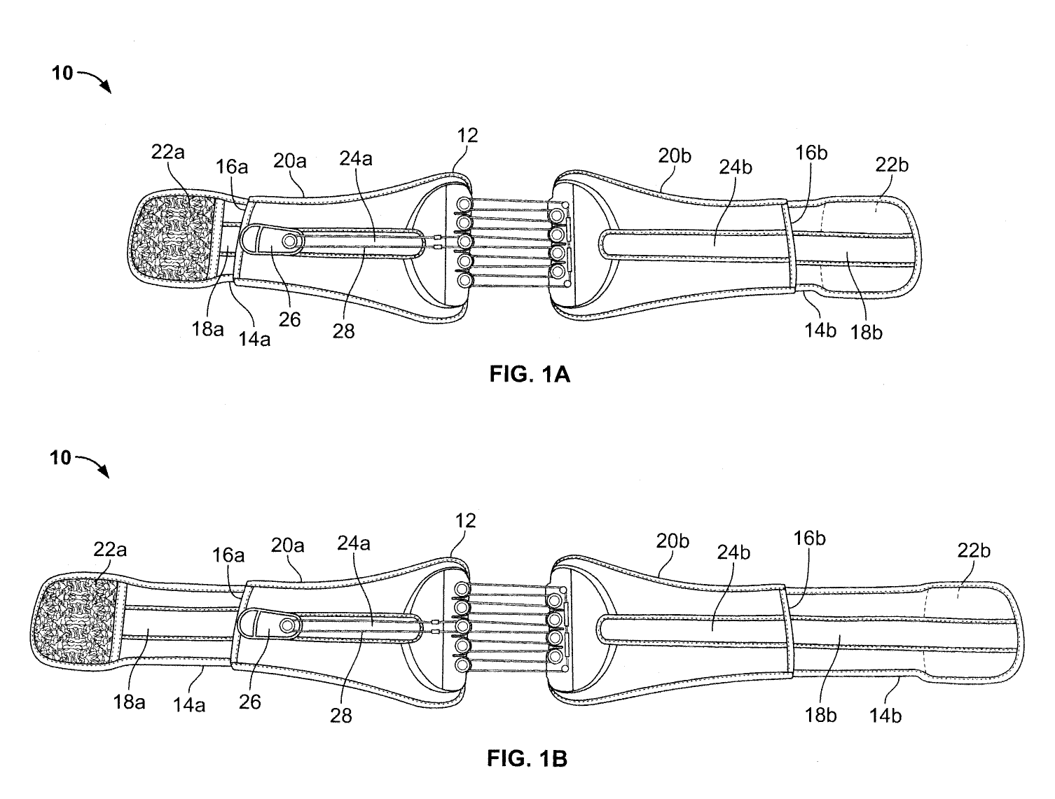

With reference to FIG. 1A, there is shown an adjustable length orthotic device 10 in accordance with an embodiment of the present invention. In this embodiment, the orthotic device 10 is a back brace. The orthotic device 10 comprises a center brace segment 12 that is configured to provide support to the torso of a user, and two end segments 14 that may be moved in and out of the center brace segment 12 to adjust the length of the orthotic device 10. The center brace segment has pockets 16 at either end for insertion of the end segments 14. In the depicted configuration, the end segments 14 are inserted deep into pockets 16 so that the length of the orthotic device 10 is not much longer than the length of the center brace segment 12. The end segments 14 may be pulled further out of the pockets to lengthen the back brace 10. This lengthened configuration is displayed in FIG. 1B, where the end segments 14 have been pulled further out of the pockets 16. By inserting the end segments 14 further into pockets 16 or pulling them out, the user is able to adjust the length of the orthotic device to fit the user's size.

The center brace segment 12 may include two brace segments 20a and 20b. Each brace segment, 20a and 20b, contains a pocket, 16a or 16b, for insertion of an end segment, 14a or 14b. The opposite end segments 14a, 14b may include complementary fastener or fastening means 22a and 22b for securing the two ends together after the orthotic device 10 is wrapped around the patient's torso. Fastening means 22b is on the reverse side of end segment 14b. Although buttons, snap fasteners, or similar fasteners may be employed to secure the free ends of the orthotic device together, a preferred embodiment uses complementary sections of hook-and-loop fastener fabric mounted on the opposite overlapping sides of the opposite end segments 14a, 14b proximate at least the free ends thereof.

Additionally, horizontal strips 24a, 24b of hook-and-loop material are arranged on the exterior surface of each body brace segment 20a, 20b to allow for removably securing a handle element 26 to one of the body brace segments. The handle element is attached to the end of a cord 28 and used in the tension adjusters of a closure system for the orthotic device 10. The other end of the cord 28 is connected to a set of pulleys, which are configured in such a way that pulling the cord 28 will pull the body brace segments 20a and 20b together and tighten the body brace 12 around the user's torso. Such a pulley system is disclosed in greater detail in U.S. Pat. No. 6,213,968 to Heinz et al. or U.S. Pat. No. 35,940 to Heinz et al., which are incorporated herein by reference. The handle element 26 may include an easily graspable member, such as a cloth tab, loop, ring or bail. The handle 26 may also include a means of detachably securing the handle to a body brace segment after adjustment has been made or the device has been removed from the wearer. Such a means of securing could include a series of clasps, a buckle and strap, or a hook-and-loop arrangement. In the closure system shown in FIGS. 1A and 1B, a piece of hook-and-loop fabric is affixed to the underside of the handle element 26. After adjustment has been made and the appropriate tension has been established in the cord 28, the handle 26 may be releasably secured to a segment 20a or 20b by placing the hook-and-loop fabric on the handle 26 in contact with a portion of the complementary hook-and-loop material 24 on the body brace segment 20a or 20b.

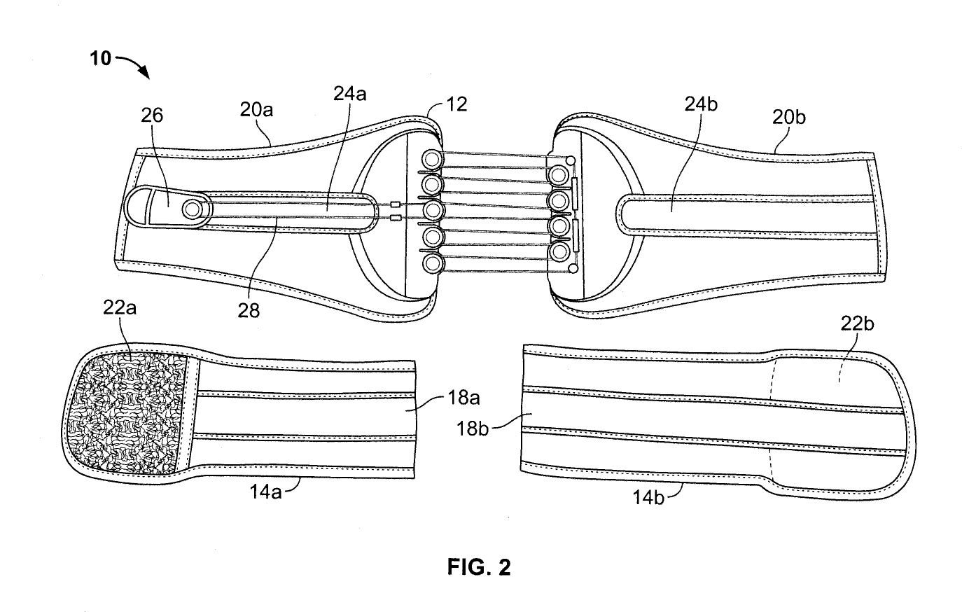

FIG. 2 depicts the adjustable length orthotic device 10 with the two end segments 14a, 14b separated from the center brace segment 12. The end segments 14a, 14b are inserted into the pockets 16a and 16b, respectively. The end segments 14a, 14b may be pulled out or inserted further in the pockets 16a, 16b to adjust the length of the orthotic device 10. The end segments 14a, 14b are secured within pockets 16a, 16b by use of securing means 18a, 18b. In this embodiment, the securing means 18a, 18b are hook and loop fastener material. Buttons, snap fasteners, or similar fasteners may also be employed. Two strips of hook and loop fastener material 18a, 18b are mounted on the end segments 14a, 14b. Corresponding strips of hook and loop fastener material are mounted on an inside wall of the pockets 16a, 16b such that when the end segments 14a and 14b are inserted into the pockets 16a and 16b, the strips of hook and loop fastener material on the end segments 18a,18b line up with the corresponding strips of hook and loop fastener material on the inside walls of pockets 16a and 16h. When the strips of hook and loop fastener come into contact with one another, the end segments 14a, 14b are held in place inside the pockets 16a, 16b. Due to the fact that the end segments 14a, 14b are secured inside the pockets 16a, 16b, there is no risk that the securing means could get caught on an object as the user walks by, a problem that is common with many prior art adjustable length orthotic device in which the securing means are placed outside the brace. Another benefit of the present embodiment is that the use of hook and loop fastener allows for the end segments to be secured at any position within the pockets, thereby allowing for the brace's length to be adjusted by any amount within the possible range, rather than requiring that adjustments be made at fixed length intervals.

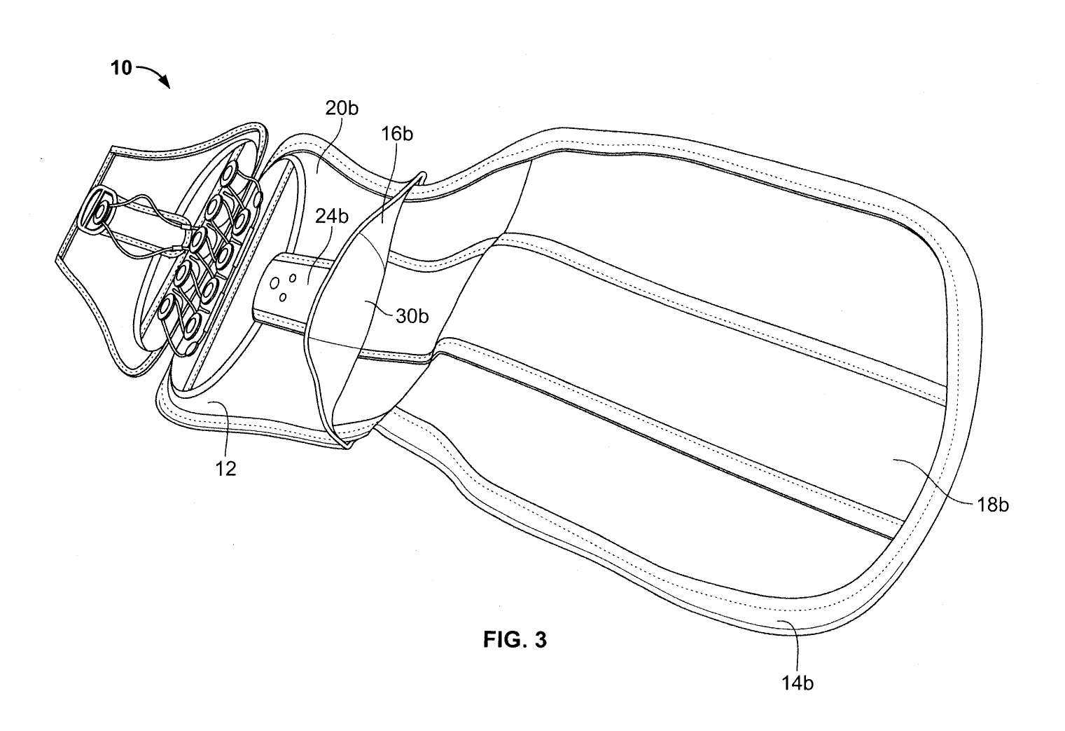

FIG. 3 depicts a side perspective view or the orthotic device 10 with the end segments 14a, 14b inserted into pockets 16a, 16b. This perspective reveals the inside of one pocket 16b with securing means 30b covering a portion of an inside wall of the pocket 16b. In this embodiment, securing means 30b is a strip of hook and loop fastener. When the pocket hook and loop fastener 30b comes into contact with the end segment hook and loop fastener 18b, it secures the end segment 14b within the pocket 16b. While only one pocket 16b is depicted, it will be understood that the other pocket 16a is a mirror image of the depicted pocket 16b. In order to adjust the length of the brace 10, the user simply undoes the securement means—e.g., by disconnecting the strips of hook and loop fastener—adjusts the position of the end segment within the pocket, and then re-secures the end segment within the pocket at the adjust position—e.g., by bringing the pocket hook and loop fastener in contact with the end segment hook and loop fastener. While the present embodiment deals with adjusting the length of a brace using hook and loop fastener as the securing means, it will be understood that other securing means may be used, which may include but are not limited to other releasable securing means such as buttons and fasteners.

Although the invention has been disclosed with reference only to the presently preferred embodiments, those of ordinary skill in the art will appreciate that various modifications can be made without departing from the invention. Accordingly, the invention is defined only by the following claims.

What is claimed is:

1. An adjustable length orthotic device comprising:
a center brace segment having a first pocket and a second pocket;
a first end segment that is configured to be inserted into the first pocket; and
a second end segment that is configured to be inserted into the second pocket,
wherein the length of the adjustable length orthotic device may be adjusted by adjusting the respective positions of the first and second end segments inside the first and second pockets, respectively.

2. The adjustable length orthotic device of claim 1, further comprising a securing means for securing the first and second end segments within the first and second pockets, respectively.

3. The adjustable length orthotic device of claim 2, wherein the securing means is hook and loop fastener.

4. The adjustable length orthotic device of claim 1, wherein the adjustable length orthotic device is a back brace.

5. A method for adjusting the length of an adjustable length orthotic device, the method comprising the steps of:
obtaining an adjustable length orthotic device comprising
a center brace segment having a first pocket and a second pocket,
a first end segment that is configured to be inserted into the first pocket, and
a second end segment that is configured to be inserted into the second pocket;
inserting the first end segment into the first pocket; and
inserting the second end segment into the second pocket.

6. The method of claim 5, wherein:
the adjustable length orthotic device further comprises securing means for releasably securing the first and second end segments within the first and second pockets, respectively;
the step of inserting the first end segment into the first pocket further comprises the step of releasably securing the first end segment within the first pocket at a first position; and
the step of inserting the second end segment into the second pocket further comprises the step of releasably securing the second end segment within the second pocket at a second position.

7. The method of claim 6 wherein the securing means is hook and loop fastener.

8. The method of claim 6 wherein the orthotic device is a back brace.

9. The method of claim 6 further comprising the steps of:
unsecuring the first end segment from the first pocket;
adjusting the first end segment's position within the first pocket;
re-securing the first end segment within the first pocket at a first adjusted position that is different from the first position;
unsecuring the second end segment from the second pocket;
adjusting the second end segment's position within the second pocket; and
re-securing the second end segment within the second pocket at a second adjusted position that is different from the second position.

10. A method for using an adjustable length orthotic device, the method comprising the steps of:
obtaining an adjustable length orthotic device comprising
a center brace segment having a first pocket and a second pocket,
a first end segment that is configured to be inserted into the first pocket,
a second end segment that is configured to be inserted into the second pocket; and
securing means for releasably securing the first and second end segments within the first and second pockets, respectively;
inserting the first end segment into the first pocket and releasably securing the first end segment within the first pocket using the securing means at a first position;
inserting the second end segment into the second pocket and releasably securing the second end segment within the second pocket using the securing means at a second position;
unsecuring the first end segment from the first pocket and releasably securing the first end segment within the first pocket using the securing means at a first adjusted position that is different from the first position; and
unsecuring the second end segment from the second pocket and releasably securing the second end segment within the second pocket using the securing means at a second adjusted position that is different from the second position.

11. The method of claim 10 wherein the securing means is hook and loop fastener.

12. The method of claim 10 wherein the orthotic device is a back brace.
